(19) United States
(12) Patent Application Publication
(10) Pub. No.: US 2023/0263555 A1
(43) Pub. Date: Aug. 24, 2023

(54) SURGICAL ASSEMBLY DESIGNED TO REDUCE A BONE FRACTURE

(71) Applicant: NEWCLIP INTERNATIONAL, Luxembourg (LU)

(72) Inventors: Julien BALLERINI, NANTES (FR); Nicolas SMITH, SYDNEY (AU); Grégoire LARCHE, CHOLET (FR); Jean-Pierre PODGORSKI, SEVREMOINE (FR)

(21) Appl. No.: 17/922,198

(22) PCT Filed: Apr. 29, 2021
(86) PCT No.: PCT/EP2021/061292

§ 371 Date: Oct. 28, 2022

(30) Foreign Application Priority Data
Apr. 29, 2020 (FR) FR2004250

(51) Int. Cl. A61B 17/80; A61B 1/317; A61B 17/88; A61B 17/86; A61B 17/90; A61B 17/84

(52) U.S. Cl. CPC A61B 17/8014; A61B 1/317; A61B 17/8057; A61B 17/888; A61B 17/864; A61B 17/90; A61B 17/846; A61B 2090/062

(57) ABSTRACT

Disclosed is a surgical assembly for reducing a fracture situated at bone epiphyses or joints including: an osteosynthesis support plate, a set of diaphyseal fixing members, a set of cannulated epiphyseal fixing members, a set of fixing pins, at least one cannulated sighting guide, a cannulated tool for rotating the fixing members and the sighting guide, also acting as a measuring instrument (for determining the length of at least some of the epiphyseal fixing members to be used), a cannulated drilling device (for drilling holes suitable for positioning at least some of the fixing members) and, preferably, an arthroscopic device designed to allow the practitioner to view some of the operative phases.

### TECHNICAL FIELD OF THE INVENTION

The present invention relates to the general field of surgical equipment, and in particular the field of devices for osteosynthesis techniques.

More particularly, it relates to a surgical assembly (or kit) for reducing a bone fracture, in particular a fracture located at the bone epiphyses or joints.

### STATE OF THE ART

Bone consolidation of certain fractures require osteosynthesis equipment or devices to be fitted on the different bone fragments, to stabilize and prevent an excessive inter-fragmentary mobility.

The surgeons then use, in most cases, osteosynthesis devices mounted by screwing on the bone fragments, such as screws, pins or screwed plates, to stabilize these bone fragments and therefore the fracture.

In particular, there exist many types of plates associated with a set of fixing screws, which are proposed to the surgeons to reduce a facture located at the bone epiphyses (distal radius, proximal humerus, proximal tibia . . . ).

The osteosynthesis support plate can then comprise an elongated body portion, extended by an integral head portion.

The body portion of the support plate (called the diaphyseal plate portion) comprises a plurality of through-holes for its fixing to the receiving bone material by means of fixing screws; and the head portion of the support plate (called epiphyseal plate portion) comprises a plurality of through-holes each adapted for accommodating an elongated fixing member, in particular for ensuring the desired reduction of the bone fracture.

Very generally, after temporary anatomical reduction of the area treated by means of pins, the pin(s) used are removed to allow locking screws to be fitted.

However, the anatomical reduction performed can evolve during the pin removal, or just after, due to lack of stability.

Therefore, generally, the current equipment and techniques at the disposal of the practitioners/surgeons do not allow an efficient or optimum temporary stabilization of the fracture before fixing with locking screws.

They do not always make it possible to obtain a great accuracy as regards the facture reduction. And they often require a long time for their implementation.

### DISCLOSURE OF THE INVENTION

In order to remedy the above-mentioned drawbacks of the state of the art, the present invention proposes a surgical assembly for reducing a bone fracture, in particular a fracture located at the bone epiphyses or joints, comprising:

an osteosynthesis support plate comprising a lower face and an upper face, said lower face being intended to be positioned against the receiving bone material, said support plate comprising an elongated body portion, called the diaphyseal plate portion, extended by an integral head portion, called the epiphyseal plate portion, said diaphyseal plate portion comprising a plurality of through-holes, called diaphyseal holes, and said epiphyseal plate portion comprising a plurality of through-holes, called epiphyseal holes, said diaphyseal holes and epiphyseal holes being each adapted for accommodating an elongated fixing member, certain at least of said epiphyseal holes comprising an internal thread,

a set of members for the fixing into the receiving bone material, intended to be inserted into said diaphyseal holes, called the diaphyseal fixing members, to fix said diaphyseal plate portion to the surface of the receiving bone material,

a set of members for the fixing into the bone, intended to be inserted into said epiphyseal holes, called the epiphyseal fixing members, adapted in particular to allow the desired reduction of the bone fracture,

means for the rotational operation of said diaphyseal fixing members and said epiphyseal fixing members, in such a way as to ensure their anchoring by screwing into the receiving bone material, said surgical assembly being characterized in that it further comprises:

at least one sighting guide of tubular shape, provided with an axial channel extending along its longitudinal axis, an end of which being provided with an external thread adapted to cooperate with the internal thread of one of said epiphyseal holes, and the other end of which has an operating recess,

a rotation operating tool, designed to cooperate with the operating recess of said at least one sighting guide, for the screwing of its threaded end into one of said epiphyseal holes, and the unscrewing thereof, said rotation operating tool being provided with an axial channel extending along its longitudinal axis,

a plurality of fixing pins, designed to cooperate with said at least one sighting guide, that is to say each adapted to be inserted into the axial channel of said at least one sighting guide to ensure the longitudinal guiding thereof and each adapted to ensure the desired, at least partial, temporary reduction of the bone fracture,

measurement means, designed to measure the driving depth of said fixing pins into the receiving bone material,

drilling equipment comprising a cannulated drilling tool provided with an axial longitudinal channel, suitable for being inserted on one of said fixing pins located through one of said epiphyseal holes and for drilling the receiving bone material, said epiphyseal fixing members being, at least for some of which, in the form of cannulated fixing members, provided with an axial longitudinal channel suitable for the passage of one of said fixing pins,

said means for rotating said epiphyseal fixing members being in the form of a cannulated operating tool provided with an axial longitudinal channel suitable for the passage of one of said fixing pins, and one end of which is structured to cooperate with a head recess of complementary shape formed in a member head of said epiphyseal fixing members.

Such a surgical assembly makes it possible in particular to position the epiphyseal fixing members whereas the fixing pins, fitted for the anatomical reduction of the fracture, remain in place.

Other non-limiting and advantageous features of the surgical assembly according to the invention, taken individually or according to all the technically possible combinations, are the following:
the diaphyseal fixing members and the epiphyseal fixing members have a same head recess in such a way that they can be rotated by the same cannulated operating tool;
said at least one sighting guide has an operating recess identical to the head recess of said epiphyseal fixing members in such a way that they can be rotated by the same cannulated operating tool;
the cannulated tool for rotating the sighting guides and/or the epiphyseal fixing members comprises said measurement means to measure the driving depth of said fixing pins into the receiving bone material;
the cannulated drilling tool comprises measurement means for determining the drilling depth with respect to the end of the fixing pin on which the drilling is made;
the surgical assembly also comprises arthroscopy equipment adapted to allow viewing in particular the desired anatomical reduction and the positioning of the fixing pins and the epiphyseal fixing members into the bone material;
according to one embodiment, the epiphyseal fixing members comprise (i) a member head provided with an external thread, said external thread being adapted to cooperate with the internal thread of the epiphyseal holes, and (ii) a member body, extending said member head, provided with (a) a proximal portion having an external thread suitable for being fixed by screwing into the receiving bone material, and (b) a distal portion with no thread;
according to another embodiment, the epiphyseal fixing members comprise (a) a member head provided with an external thread, said external thread being adapted to cooperate with the internal thread of the epiphyseal holes, and (b) a member body, extending said member head, said member body being provided with a thread over its whole length;
according to still another embodiment, the epiphyseal fixing members comprise (a) a member head provided with an external thread, said external thread being adapted to cooperate with the internal thread of the epiphyseal holes, and (b) a member body, extending said member head, said member body being smooth, devoid of thread;
the surgical assembly comprises a tool of the drill or surgical motor type, suitable for driving the fixing pins into the receiving bone material;
the surgical assembly comprises a first type of sighting guide of length L1, and at least one second type of sighting guide of length L2 different from L1.

Obviously, the different features, alternatives and embodiments of the invention can be associated with each other according to various combinations, insofar as they are not incompatible or exclusive with respect to each other.

### DETAILED DESCRIPTION OF THE INVENTION

Moreover, various other features of the invention emerge from the appended description made with reference to the drawings that illustrate non-limiting embodiments of the invention, and wherein:

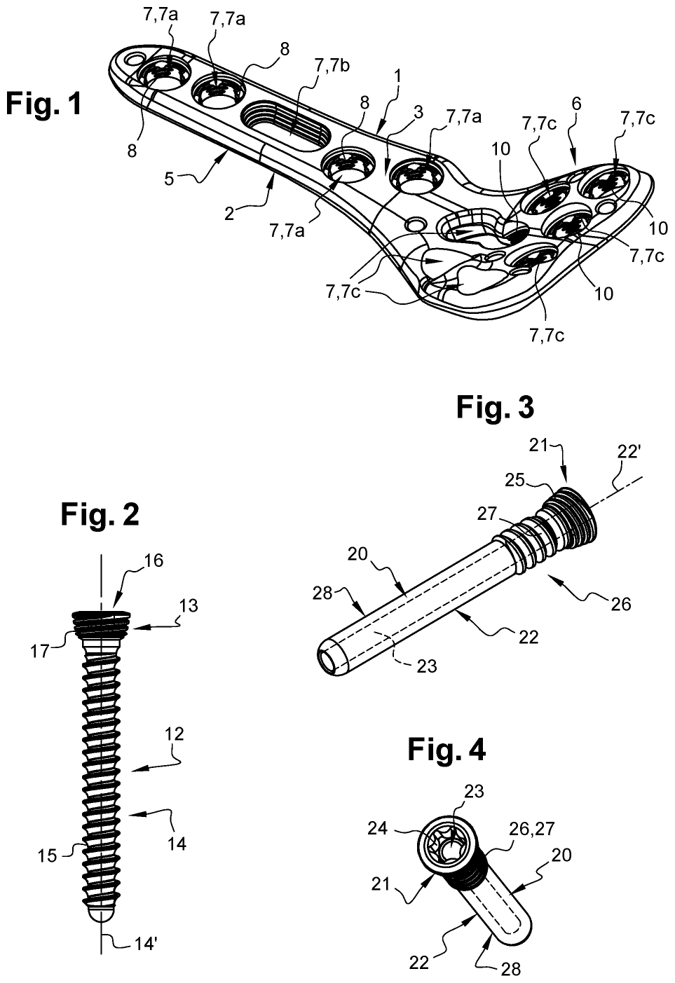

FIG. 1 is a perspective view of an osteosynthesis support plate belonging to the proposed surgical assembly;

FIG. 2 is a side view of a possible embodiment of a diaphyseal fixing member belonging to the proposed surgical assembly;

FIG. 3 is a perspective view, according to a first angle, of a possible embodiment of an epiphyseal fixing member belonging to the proposed surgical assembly;

FIG. 4 shows the epiphyseal fixing member of FIG. 3, in a perspective view according to a second angle;

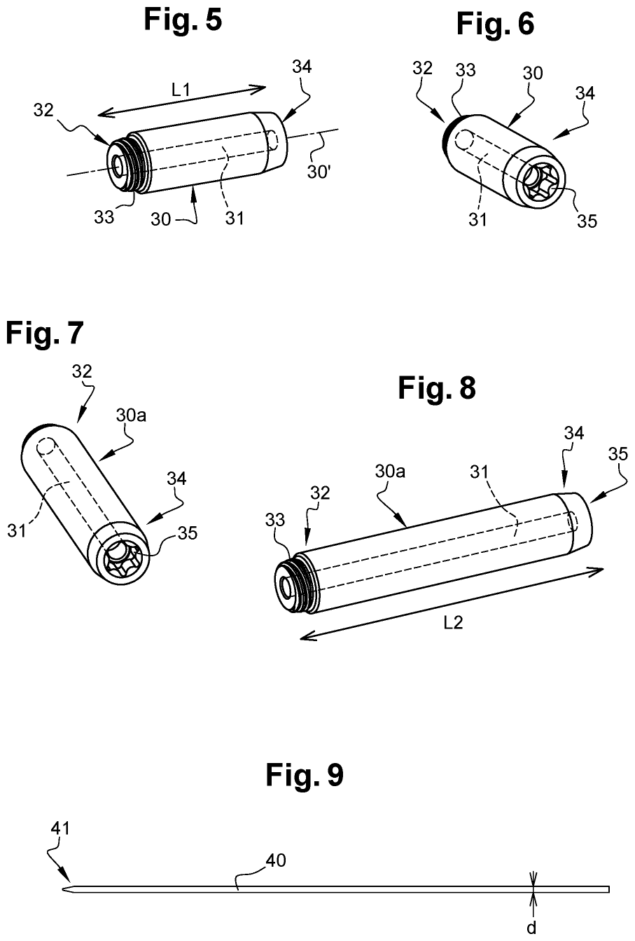

FIG. 5 is a perspective view, according to a first angle, of a first embodiment of a sighting guide belonging to the proposed surgical assembly;

FIG. 6 shows the sighting guide of FIG. 5, in a perspective view according to a second angle;

FIG. 7 is a perspective view, according to a first angle, of a second embodiment of a sighting guide belonging to the proposed surgical assembly;

FIG. 8 shows the sighting guide of FIG. 7, in a perspective view according to a second angle;

FIG. 9 is a side view of a fixing pin belonging to the proposed surgical assembly;

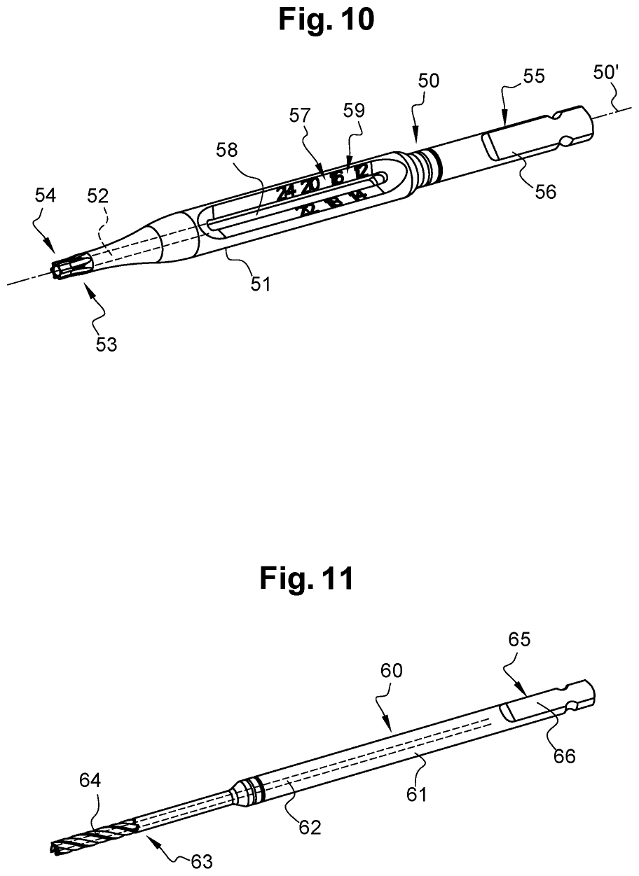

FIG. 10 is a perspective view of a possible embodiment of a cannulated operating and measurement tool belonging to the proposed surgical assembly;

FIG. 11 is a perspective view of a possible embodiment belonging of a cannulated drilling tool belonging to the proposed surgical assembly;

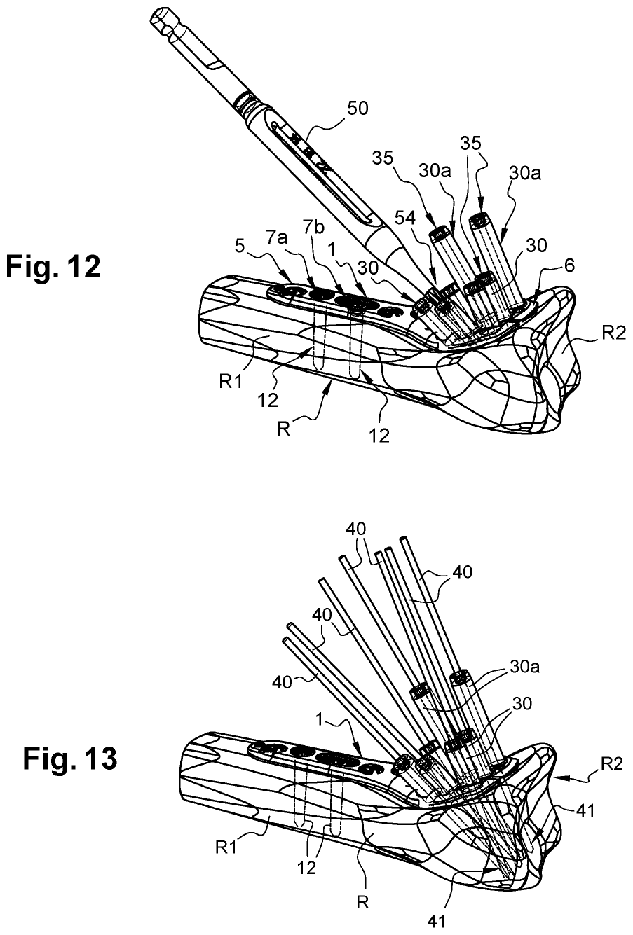

FIG. 12 is a perspective view illustrating the operation of positioning the sighting guides on the epiphyseal portion of the support plate fixed to the receiving bone material;

FIG. 13 is a perspective view illustrating the operation of positioning the fixing pins on the epiphyseal portion of the support plate;

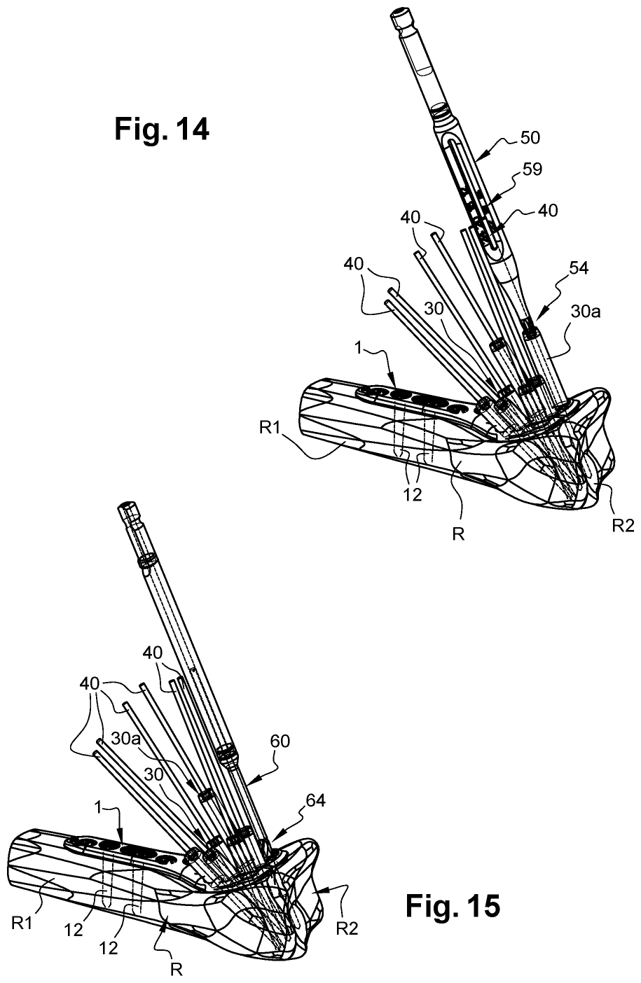

FIG. 14 is a perspective view illustrating the operation of depth measurement to define the length of an epiphyseal fixing member to be used to reduce the bone fracture, followed with the operation of removing the associated sighting guide;

FIG. 15 is a perspective view illustrating the operation of drilling for positioning an epiphyseal fixing member;

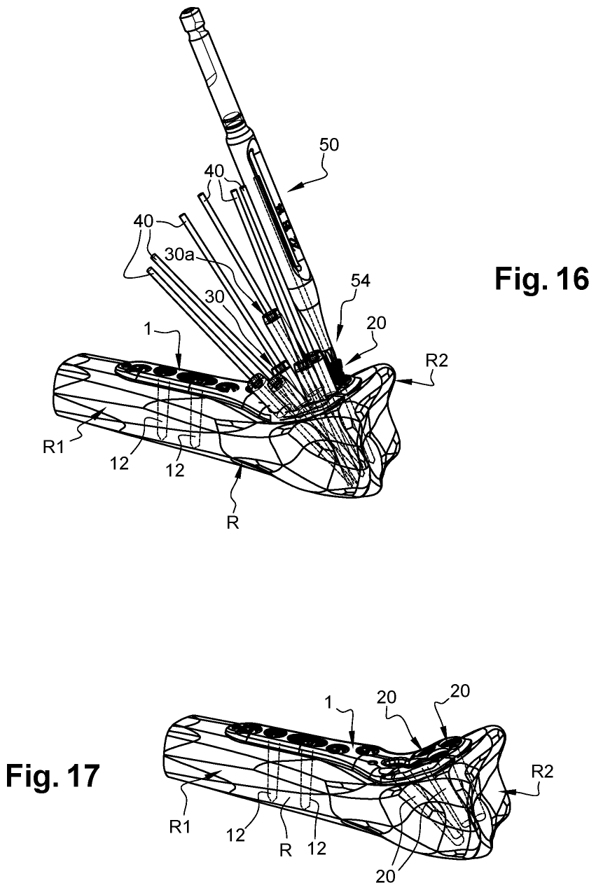

FIG. 16 is a perspective view illustrating the operation of positioning an epiphyseal fixing member;

FIG. 17 is a perspective view showing the support plate equipped with all the epiphyseal fixing members required for reducing the bone fracture in presence;

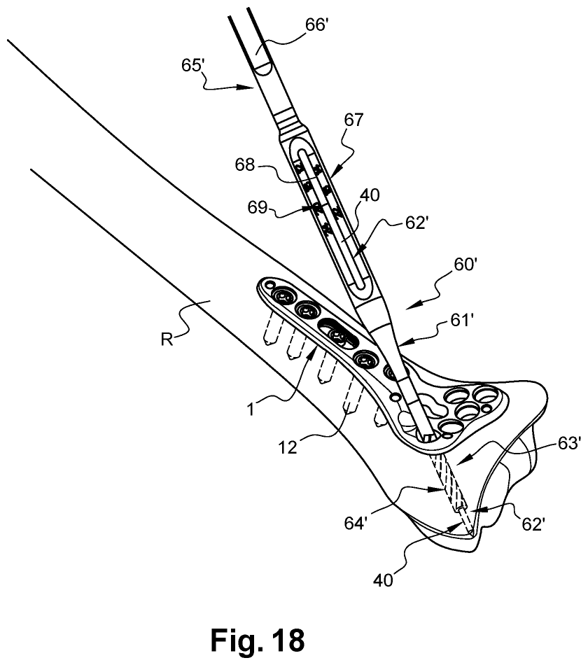

FIG. 18 is a perspective view illustrating the operation of drilling for positioning an epiphyseal fixing member, by means of an alternative embodiment of a cannulated drilling tool including measurement means suitable for determining the drilling depth.

### SURGICAL ASSEMBLY

As illustrated in the appended figures, the surgical assembly according to the invention that is proposed to the practitioner/surgeon for reducing a facture located at the bone epiphyses (in particular, a comminuted fracture), comprises in particular an osteosynthesis support plate, a set of diaphyseal fixing members, a set of epiphyseal fixing members, a set of fixing pins, a set of sighting guides, a tool (or ancillary) for rotating said fixing members and said sighting guides, also acting as a measurement ancillary (to determine the length of at least certain of the fixing members to be used), as well as drilling equipment (for the drilling of holes suitable for the positioning of at least certain of the fixing members).

Preferably, this surgical assembly also comprises arthroscopy equipment (not shown) suitable for the practitioner/surgeon to be able to view the desired anatomical reduction of the joint area, and also the current implantation of the fixing pins, the making of drilling operations and the positioning of the fixing members, in particular the epiphyseal fixing members.

The osteosynthesis support plate 1 illustrated in isolation in FIG. 1 is a few millimetres thick and is delimited by a lower face 2 intended to come into contact with the receiving bone material and by an opposite upper face 3.

This support plate 1 comprises an elongated body portion 5, called the diaphyseal plate portion 5, suitable to be positioned on the diaphysis of the receiving bone material, and an integral head portion 6, called the epiphyseal plate portion 6, adapted to be positioned on the bone epiphysis, at the fracture to be reduced.

The support plate 1 is provided with a plurality of holes 7 through its thickness, between its lower face 2 and its upper face 3, that are intended for the passage of fixing members, for its fixing to the receiving bone material and, also, for some of them, for the desired reduction of the bone fracture.

The diaphyseal plate portion 5 comprises a plurality of diaphyseal holes 7a, 7b intended for the passage of diaphyseal fixing members for the fixing of the support plate 1 on the reception bone material.

Here, four of these diaphyseal holes 7a have a generally cylindrical shape. Preferably, these diaphyseal holes 7a comprise an internal thread 8 suitable for cooperating with a complementary thread formed on the head of the fixing members intended to be associated thereto.

Here again, one of these diaphyseal holes 7b has an oblong elongated shape. This oblong diaphyseal hole 7b is used to adjust the positioning of the support plate 1 on the diaphysis of the bone before the positioning of the fixing members into the cylindrical diaphyseal holes 7a.

The epiphyseal plate portion 6 comprises a plurality of epiphyseal holes 7c here arranged along two lines substantially perpendicular to the axis of the diaphyseal plaque portion 5. In the exemplary embodiment shown, the distal (end) line is composed of an alignment of four holes 7c whereas the proximal line is composed of an alignment of three holes 7c.

Here, these epiphyseal holes 7c have a generally cylindrical shape. Preferably, these diaphyseal holes 7c comprise an internal thread 10 suitable for cooperating with a complementary thread formed on the head of the fixing members intended to be associated thereto.

A possible embodiment of a diaphyseal fixing member 12 is illustrated in FIG. 2.

This diaphyseal fixing member 12 is in the form of a fixing screw comprising—a screw head 13, at least a portion of the contour of which is intended to come into contact with a portion of the periphery of one of the diaphyseal holes 7a, 7b, and—a screw body 14, of longitudinal axis 14′, provided with a thread 15 for its anchoring into the receiving bone material.

A hollow head recess 16 (or operating recess 16), made in the screw head 13, allows the fixing screw 12 to be rotated by means of a suitable tool, for its implantation into the receiving bone material. The operating recess 16 is arranged axially at the end of the fixing member 12.

The periphery of the screw head 13 comprises an external thread 17 intended to cooperate with the internal thread 8 of the diaphyseal holes 7a to obtain a locking of the fixing screw 12 on the support plate 1.

A possible embodiment of an epiphyseal fixing member 20 is illustrated in FIGS. 3 and 4.

This epiphyseal fixing member 20 is in the form of a cannulated elongated member, that is to say provided with an axial longitudinal channel (this axial channel being adapted to the passage of a fixing pin, as described hereinafter in the description).

More precisely, this epiphyseal fixing member 20 comprises—a member head 21, at least a portion of the contour of which is intended to come into contact with a portion of the periphery of one of the epiphyseal holes 7c, and—a member body 22, of longitudinal axis 22′.

An axial longitudinal channel 23 extends over the whole length of the epiphyseal fixing member 20 and exits at the two ends thereof, that is to say at the member head 21 and at the free end of the member body 22.

A hollow head recess 24 (or operating recess 24), made in the screw head 21, allows the fixing member 20 to be rotated by means of a suitable tool, for its implantation into the receiving bone material. The operating recess 24 is arranged axially at the end of the fixing member 20.

Advantageously, the head recess 16 of the diaphyseal fixing members 12 and the head recess 24 of the epiphyseal fixing members 20 are identical in such a way that these two types of fixing members 12, 20 can be rotated by the same operating tool.

The periphery of the screw head 21 comprises an external thread 25 intended to cooperate with the internal thread 10 of the diaphyseal holes 7c to obtain a locking of the fixing member 20 on the support plate 1.

In the embodiment illustrated, the member body 22 is provided (a) with a proximal portion 26, located on the side of the member head 21, which has an external thread 27 suitable for being fixed by screwing into the bone material, and (b) a distal portion 28 devoid of thread up to its free end. Here, the length of the threaded proximal portion 26 is comprised between ⅕ and ⅓ of the length of the member body 22.

In an alternative embodiment, the whole length of the member body 22 of the fixing members 20 has an external thread.

And according to still another alternative embodiment, the member body 22 of the fixing members 20 is smooth and has thus no external thread.

A possible embodiment of a sighting guide 30 for a surgical assembly according to the invention is illustrated in FIGS. 5 and 6.

This sighting guide 30 has the general shape of a tube of longitudinal axis 30′, provided with an axial hole or channel 31.

An end 32 of this sighting guide 30 is provided with an external thread 33 suitable to cooperate with the internal thread 10 of one of the epiphyseal holes 7c. On the other hand, the other end 34 (opposite to the end 32) of the sighting guide 30 has an operating recess 35 to allow it to be rotated about the longitudinal axis 30′ by means of a suitable operating tool. The operating recess 35 is arranged axially at the end of the sighting guide 30.

This operating recess 35 is identical to the head recess 16 of the diaphyseal fixing members 12 and to the head recess 24 of the epiphyseal fixing members 20 in such a way that they can be rotated by the same operating tool.

FIGS. 7 and 8 illustrate an alternative embodiment of a sighting guide 30a for a surgical assembly according to the invention. This sighting guide 30a is identical to the sighting guide 30 describes in relation with FIGS. 5 and 6, except at regards its length L2 that is longer than length L1 of the sighting guide 30 described hereinabove.

A possible embodiment of a fixing pin 40 for a surgical assembly according to the invention is illustrated in FIG. 9.

This fixing pin 40 is made of metal, for example stainless steel or titanium. It is in the form of a smooth rod including a tip-shaped end 41; its length may be between 40 and 200 mm and its diameter d, which is constant, may be between 0.8 and 2.5 mm.

This diameter d of the fixing pin 40 corresponds, within a clearance, to the diameter of the axial longitudinal channel 23 of the epiphyseal fixing member 20 and to the diameter of the axial channel 31 of the sighting guides 30, 30a.

A possible embodiment of a cannulated operating tool 50 for a surgical assembly according to the invention is illustrated in FIG. 10.

This operating tool 50 comprises an elongated body 51, of longitudinal axis 50′, provided with an axial channel 52. One of its ends 53 has an operating head 54 and its other end 55 comprises means 56 suitable for its removable fixing to an operating handle. The operating head 54 is arranged axially at the end 53 of the fixing member 50.
The operating head 54 is designed so as to be able to cooperate with the operating recess 35 of the sighting guides 30, 30a, with the head recess 24 of the epiphyseal fixing members 20 and with the head recess 16 of the diaphyseal fixing members 12.
On the other hand, the diameter of the axial channel 52 corresponds, within a clearance, to the diameter d of the fixing pins 40.

Between these two ends 53 and 56, the cannulated operating tool 50 includes measurement means 57 suitable for measuring the driving depth of the fixing pin 40 into the receiving bone material R, as explained hereinafter in the description.

These measurement means 57 comprise an elongated aperture 58 offering a visual access to a portion of the axial channel 52, associated with a graduation system 59.
This graduation system 59 is designed, once the operating tool 50 positioned on a fixing pin 40 driven into the bone material R, with its operating head 54 meshed with the operating recess of an associated sighting guide 30, 30a, to indicate to the practitioner/surgeon the driving depth of the fixing pin 40, by the position of the free end of this pin 40 opposite said graduation system 59, or by means of a marking line made over the length of the fixing pin 40. And the driving length of the fixing pin 40 indicates to the practitioner/surgeon the length of the epiphyseal fixing member 20 to be used for the fixing into the bone.

Therefore, it is understood that this cannulated operating tool 50 fulfils several functions: one for rotating the sighting guides and the fixing members, and another for measuring the driving depth of the fixing pins.

In alternative embodiments, several operating tools can be provided, for example if the operating recesses 16, 24 and 35 are different; or if it is desired to have a depth measurement tool separate from the rotation operating tool.

A possible embodiment of a cannulated drilling tool 60 for a surgical assembly according to the invention is illustrated in FIG. 11.

This canulated drilling tool 60 consists of a drill bit intended to cooperate with a rotary drive tool, such as a surgical motor or a drill (non shown) to form together drilling equipment.
The canulated drilling tool 60 has an elongated body 61 provided with an axial longitudinal hole or channel 62. One of its ends 63 comprises a drilling head 64 and its other end 65 comprises means 66 suitable for being fixed on the associated drill (or surgical motor) head, in such a way as to ensure the driving in rotation of the drilling head 64.
The diameter of the axial longitudinal channel 62 corresponds, within a clearance, to the diameter d of the fixing pins 40.

The surgical assembly according to the invention also advantageously comprises a tool suitable for driving the fixing pins 40 into the bone material R. This tool may consist of a drill (or a surgical motor).

And according to still another feature, this surgical assembly may also comprise arthroscopy equipment adapted to allow the practitioner/surgeon to view directly in particular the anatomical reduction of the fracture, and also the positioning of the fixing pins 40 and the epiphyseal fixing members 20 into the bone material R.

The arthroscopy equipment, not shown and conventional per se, comprises a miniaturized camera at the end of an optical fibre connected to a screen.

To allow the implementation of a surgical operation adapted to the facture in presence, the surgical assembly according to the invention that is proposed to the practitioner comprises:

a plurality of osteosynthesis support plates 1 that are different from each other by their general shape and/or their size,

a plurality of diaphyseal 12 and epiphyseal 20 fixing members of different shapes and/or sizes,

at least one sighting guide 30, 30a, and preferably several sighting guides 30, 30a (for some of them, identical to each other, and for others, of different length),

several fixing pins 40 of identical length or not,

at least one tool 50 for measuring the length of the epiphyseal fixing members 20 to be placed, and for rotating the different fixing members 12, 20 as well as the sighting guides 30, 30a,

a set of drilling tools 60,

a tool of the drill or surgical motor type, for the drilling tools 60 and to drive the fixing pins 40 into the receiving bone material R,

arthroscopy equipment for viewing the anatomical reduction of the fracture, and also the positioning in particular of the fixing pins 40, the epiphyseal fixing members 20 and the diaphyseal fixing members 12,

potentially, a set of drilling guides.

Operating Mode

FIGS. 12 to 17 illustrate in different steps the operating mode for reducing a bone fracture by means of a surgical assembly according to the invention as described hereinabove.

FIG. 12 illustrates a bone material R (for example, the end of a radius) comprising a diaphyseal portion R1 and an epiphyseal portion R2 having undergone a bone fracture that is desired to be reduced.

In a first time, the diaphyseal portion 5 of an osteosynthesis support plate 1 is fixed to the diaphyseal portion R1 of the bone material R by means of diaphyseal fixing screws 12, in such a way that the epiphyseal plate portion 6 is located opposite the bone fracture to be reduced.

In the exemplary embodiment shown, a diaphyseal fixing screw 12 is implanted into the bone material R via the oblong diaphyseal hole 7b, and another diaphyseal fixing screw 12 is implanted into the bone material R via one of the cylindrical diaphyseal holes 7a. All the cylindrical diaphyseal holes 7a can be equipped with a diaphyseal fixing screw 12 to optimize the fixing of the osteosynthesis support plate 1.

These diaphyseal fixing screws 12 are positioned by means of a screwdriver by cooperation of its operating head with the head recess 16 of said diaphyseal screws 12.

Once the epiphyseal plate portion 6 correctly positioned opposite the bone fracture to be reduced, the practitioner positions the sighting guides 30, 30a on this epiphyseal plate portion 6. For that purpose, the external thread 33 of the distal end 32 of the sighting guides 30, 30a is screwed on the internal thread 10 of the epiphyseal holes 7c chosen. This screwing may be made using the canulated operation tool 50 (whose operating head 54 cooperates with the operating recess 35 of the sighting guides 30, 30a).

As can be seen in FIG. 13, the practitioner then positions the fixing pins 40 using the sighting guides 30, 30a.

Each fixing pin 40 passes in the axial channel 31 of one of the sighting guides 30, 30a and is guided by the latter. The driving of the fixing pins 40 is made by the suitable tool of the drill (or surgical motor) type; and this driving can be made under arthroscopy in such a way that the practitioner/surgeon can view precisely the positioning of each fixing pin 40.

The driving of the fixing pins 40 is made until their tip-shaped end 41 reaches the opposite cortex of the bone. This operation of positioning the fixing pins 40 may be made under arthroscopy.

Then, as shown in FIG. 14, the practitioner positions the canulated operating and measurement tool 50 on a fixing pin 40, with its associated sighting guide 30, 30a still in place, to implement its function of “depth measurement”, in order to measure the driving depth of this fixing pin, in such a way as to know the length of the epiphyseal fixing member 20 that will have to be used.

For that purpose, as mentioned hereinabove, the practitioner positions the operating tool 50 on the fixing pin 40 driven into the bone material R, with its operating head 54 meshed with the operating recess 35 of the sighting guide 30, 30a; and the graduation system 59 associated with the elongated aperture 58, opposite the positioning of the free end of the fixing pin 40 (or opposite a marking line of the length of this pin) indicates to the practitioner the driving depth of the pin 40, and hence the required length for the epiphyseal fixing member 20 to be used thereafter.

The practitioner then removes the sighting guide 30, 30a by means of the canulated operating tool 50 (this removal is made without affecting the positioning of the associated fixing pin 40). And he can then drill the receiving hole of the epiphyseal fixing member 20 by means of the drilling equipment comprising the canulated drilling tool 60, as illustrated in FIG. 15.

Preferably, this drilling operation is implemented under arthroscopy; it is guided by the fixing pin 40 and it is made until reaching the opposite cortex (preferably without passing through this cortex).

The end of the fixing pin 40 preferably remains driven and held by this cortex.

Once the drill performed, the epiphyseal fixing member 20 chosen may be implanted by being driven and/or screwed into the obtained hole, as can be seen in FIG. 16. This operation is made by driving and screwing by means of the canulated operating tool 50, the epiphyseal fixing member 20 being guided by the fixing pin 40.

The epiphyseal fixing member 20 is locked in position by screwing of the external thread 25 of its member head 21 in the internal thread 10 of the associated epiphyseal hole 7c. The completion of this screwing can be done, after removal of the fixing pin 40, by means of a solid (not canulated) screwdriver.

This operation of positioning the epiphyseal fixing member 20 may be made under arthroscopy.

All the pins required for the reduction of the fracture can be positioned. Thereafter, the above-mentioned operations of depth measurement (FIG. 14), removal of the sighting guide 30, 30a, drilling (FIG. 15), and positioning of the epiphyseal fixing member 20 (FIG. 16), are made for each of the fixing pins 40, to obtain the osteosynthesis support plate 1 equipped with all the epiphyseal fixing members 20 required for reducing the bone fracture in presence, as illustrated in FIG. 17.

By way of alternative, a single sighting guide 30, 30a can be proposed in the surgical assembly according to the invention, this single sighting guide being reused after the positioning of each fixing pin 40 and the positioning of an epiphyseal fixing member 20.

However, preferably, several sighting guides 30, 30a are used, wherein the longest of which can be used to space apart certain tissues, nerves or others, during their positioning on the osteosynthesis plate, in order to facilitate the access to the bone area to be treated.

Generally, such a surgical assembly makes it possible to facilitate and optimize the temporary stabilization of a joint fracture.

A possible alternative embodiment of a cannulated drilling tool 60′ for a surgical assembly according to the invention is illustrated in FIG. 18.

This canulated drilling tool 60′ consists of a canulated drill bit for drilling the bone material on a fixing pin 40 in place, and that is equipped with measurement means adapted to inform the practitioner/surgeon about the drilling depth with respect to the end of the pin.

The canulated drilling tool 60′ has an elongated body 61′ provided with an axial longitudinal hole or channel 62′. One of its ends 63′ comprises a drilling head 64′ and its other end 65′ comprises means 66′ suitable for being fixed on the associated drill (or surgical motor) head, in such a way as to ensure the driving in rotation of the drilling head 64′.

The diameter of the axial channel 62′ corresponds, within a clearance, to the diameter of the fixing pins 40.

Between its two ends 63′ 65′, the cannulated drilling tool 60′ comprises the above-mentioned measurement means, marked 67, suitable for informing the practitioner about the drilling depth with respect to the end of the pin 40 on which the drilling is made.

These measurement means 67 comprise an elongated aperture 68 offering a visual access to a portion of the axial channel 62′, associated with a graduation system 69.

This graduation system 69 is designed to indicate to the practitioner/surgeon the current drilling depth with respect to the end of the pin 40 driven into the bone material, by the position of the free end of this pin 40 opposite said graduation system 69, or by means of a marking line made over the length of the fixing pin 40.

For example, the practitioner/surgeon can choose to perform the drill up to the tip of the pin 40, or just a little before arriving at this pin tip.

It is therefore comprised that such a canulated drilling tool 60′ makes it possible to ensure a drilling for the positioning of the fixing members, and also to determine the drilling depth with respect to the driven end of the fixing pins 40.

1. A surgical assembly for reducing a bone fracture, in particular a fracture located at the bone epiphyses, comprising:
an osteosynthesis support plate comprising a lower face and an upper face, said lower face being intended to be positioned against the receiving bone material, said support plate comprising an elongated body portion, called the diaphyseal plate portion, extended by an integral head portion, called the epiphyseal plate portion, said diaphyseal plate portion comprising a plurality of through-holes, called diaphyseal holes, and said epiphyseal plate portion comprising a plurality of through-holes, called epiphyseal holes, said diaphyseal holes and epiphyseal holes each adapted for accommodating an elongated fixing member, certain at least of said epiphyseal holes comprising an internal thread,
a set of members for the fixing into the bone material intended to be inserted into said diaphyseal holes, called the diaphyseal fixing members, to fix said diaphyseal plate portion to the surface of the bone material,
a set of members for the fixing into the bone material, intended to be inserted into said epiphyseal holes, called the epiphyseal fixing members, adapted in particular to allow the desired reduction of the bone fracture,
means for rotating said diaphyseal fixing members and said epiphyseal fixing members, in such a way as to ensure their anchoring by screwing into the bone material,
the surgical assembly further comprising:
at least one sighting guide of tubular shape, provided with an axial channel extending along its longitudinal axis, an end of which being provided with an external thread adapted to cooperate with the internal thread of one of said epiphyseal holes, and the other end of which has an operating recess,
a rotation operating tool, designed to cooperate with the operating recess of said at least one sighting guide, for the screwing of its threaded end into one of said epiphyseal holes, and the unscrewing thereof, said rotation operating tool being provided with an axial channel extending along its longitudinal axis,
a plurality of fixing pins, designed to cooperate with said at least one sighting guide, that is to say each adapted to be inserted into the axial channel of said at least one sighting guide to ensure the longitudinal guiding thereof and each adapted to ensure the desired, at least partial, temporary reduction of the bone fracture,
measurement means, designed to measure the driving depth of said fixing pins into the receiving bone material,
drilling equipment comprising a cannulated drilling tool provided with an axial longitudinal channel, suitable for being inserted on one of said fixing pins positioned through one of said epiphyseal holes and for drilling the bone material,
said epiphyseal fixing members being, at least for some of them, in the form of cannulated fixing members, provided with an axial longitudinal channel suitable for the passage of one of said fixing pins,
said means for rotating said epiphyseal fixing members being in the form of a cannulated operating tool provided with an axial longitudinal channel suitable for the passage of one of said fixing pins, and one end of which is structured to cooperate with a head recess of complementary shape formed in a member head of said epiphyseal fixing members.

2. The surgical assembly according to claim 1, wherein said diaphyseal fixing members and said epiphyseal fixing members have a same head recess in such a way that they can be rotated by the same cannulated operating tool.

3. The surgical assembly according to claim 1, wherein said at least one sighting guide has an operating recess identical to the head recess of said epiphyseal fixing members in such a way that they can be rotated by the same cannulated operating tool.

4. The surgical assembly according to claim 1, wherein the cannulated tool for rotating the sighting guides and/or the epiphyseal fixing members comprises said measurement means to measure the driving depth of said fixing pins into the receiving bone material.

5. The surgical assembly according to claim 1, wherein the cannulated drilling tool comprises measurement means for determining the drilling depth with respect to the end of the fixing pin on which the drilling is made.

6. The surgical assembly according to claim 1, further comprising arthroscopy equipment adapted to allow viewing in particular the desired anatomical reduction and the positioning of the fixing pins and the epiphyseal fixing members into the bone material.

7. The surgical assembly according to claim 1, wherein epiphyseal fixing members comprise—a member head provided with an external thread, said external thread being adapted to cooperate with the internal thread of the epiphyseal holes, and a member body, extending said member head, provided with a proximal portion having an external thread suitable for being fixed by screwing into the bone material, and a distal portion with no thread.

8. The surgical assembly according to claim 1, wherein said epiphyseal fixing members comprise—a member head provided with an external thread, said external thread being adapted to cooperate with the internal thread of the epiphyseal holes, and—a member body, extending said member head, said member body being provided with a thread over its whole length.

9. The surgical assembly according to claim 1, wherein said epiphyseal fixing members comprise—a member head provided with an external thread, said external thread being adapted to cooperate with the internal thread of the epiphyseal holes, and—a member body, extending said member head, said member body being smooth, devoid of thread.

10. The surgical assembly according to claim 1, further comprising a tool of the drill or surgical motor type, suitable for driving said fixing pins into the bone material.

11. The surgical assembly according to claim 1, further comprising a first type of sighting guide of length, and at least one second type of sighting guide of length different from.

12. The surgical assembly according to claim 2, wherein said at least one sighting guide has an operating recess identical to the head recess of said epiphyseal fixing members in such a way that they can be rotated by the same cannulated operating tool.
